(19) United States
(12) Patent Application Publication
(10) Pub. No.: US 2023/0407270 A1
(43) Pub. Date: Dec. 21, 2023

(54) POLYPEPTIDE COMPOSITIONS AND METHODS FOR USE

(71) Applicant: Totus Medicines Inc., Emeryville, CA (US)

(72) Inventors: Neil Sonin DHAWAN, Somerville, MA (US); James Abellera BLAIR, Arlington, MA (US)

(21) Appl. No.: 18/251,117

(22) PCT Filed: Nov. 2, 2021
(86) PCT No.: PCT/US2021/057712

§ 371 Date: Apr. 28, 2023

Related U.S. Application Data
(60) Provisional application No. 63/108,548, filed on Nov. 2, 2020.

(51) Int. Cl. C12N 9/02; C12Q 1/66

(52) U.S. Cl. CPC C12N 9/0069; C12Y 113/12013; C12Q 1/66; C07K 2319/61

(57) ABSTRACT

This disclosure generally relates to synthetic bioluminescent proteins such as synthetic luciferase polypeptides. Methods of making and using the proteins are also provided herein.

### CROSS REFERENCE TO RELATED APPLICATIONS

This application claims priority to U.S. Provisional Patent Application No. 63/108,548 filed Nov. 2, 2020, entitled “POLYPEPTIDE COMPOSITIONS AND METHODS FOR USE”, the contents of which are herein incorporated by reference in its entirety.

### REFERENCE TO SEQUENCE LISTING

The present application is being filed along with a Sequence Listing in electronic format. The sequence listing filed, entitled 2161-1003PCT_SEQLST.txt, was created on Nov. 2, 2021, and is 187,378 bytes in size. The information in electronic format of the Sequence Listing is incorporated herein by reference in its entirety.

### FIELD OF THE DISCLOSURE

This disclosure generally relates to bioluminescent proteins and their methods of use.

### BACKGROUND

The use of bioluminescent proteins for drug screening has demonstrated enormous interest over the past two decades (Wilson and Hastings 1998, Fan and Wood 2007, England, Ehlerding et al. 2016). They are used extensively in the investigation of cellular physiology, gene expression, protein-protein interactions, analyte detection, drug screening, and multiple high-throughput techniques. A great deal of investment in the field has focused on generating luciferase polypeptides that are most suitable for drug screening. These efforts have largely focused on approaches to make the protein smaller, more robust, stable, and environmentally insensitive. These efforts have especially focused on approaches that limit protein denaturation and artificial effects. Luciferases that use luciferin and luciferin analogues as substrates are widely used systems due to their brightness and acceptance in whole cell applications. Firefly luciferase and various beetle luciferases, for example, produce luminescence in the presence of luciferin, magnesium ions, oxygen, and ATP. However, current luciferase polypeptides are still limited by their inability to tolerate harsh experimental conditions. There remains a need for modified luciferase polypeptides.

### SUMMARY

In some embodiments, the present disclosure provides a polypeptide including an amino acid sequence I: MVFTLEDFVGDWRQTAGYNLDQVLEQGGVSSLFQNLGVSVTPIQX₁IVLSGENGLX₂I DIHVIIPYEGLSGDQMGQIEX₃IFX₄VVYPVDDHHFKVILHYGTLVIDGVTPNMIDYFG RPYEGIAVFDGX₅X₆ITVTGTLWNGNX₇IIDERLINPDGSLLFRVTINGVTGWRLX₈ERIL A, wherein X₁, X₂, X₃, X₄, X₅, X₆, and X₇ are independently R or K; X₈ is selected from the group consisting of A, R, N, D, E, Q, G, H, I, L, K, M, F, P, S, T, W, Y and V; and optionally comprising an amino acid sequence X₉ERILA attached to rightmost A residue, wherein X₉ is selected from the group consisting of A, R, N, D, E, Q, G, H, I, L, K, M, F, P, S, T, W, Y and V. X₁ may be R. X₁ may be K. X₂ may be R. X₂ may be K. X₃ may be R. X₃ may be K. X₄ may be R. X₄ may be K. X₅ and X₆ may both be R. X₅ and X₆ may both be K. X₇ may be R. X₇ may be K.

In some embodiments, the present disclosure provides a polypeptide including an amino acid sequence II:

MVFTLEDFVGDWRQTAGYNLDQVLEQGGVSSLFQNLGVSVTPIQRIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V. The polypeptide may be selected from the group consisting of SEQ ID NOs 1 to 19. The polypeptide may be selected from the group consisting of SEQ ID NO: 6, SEQ ID NO: 8, SEQ ID NO: 9, SEQ ID NO: 10, SEQ ID NO: 13, SEQ ID NO: 15, SEQ ID NO: 16, SEQ ID NO: 17, SEQ ID NO: 18 and SEQ ID NO: 19. The polypeptide may be selected from the group consisting of SEQ ID NO:8, SEQ ID NO: 10, SEQ ID NO: 16, SEQ ID NO: 17, SEQ ID NO: 18 and SEQ ID NO:19.

In some embodiments, the present disclosure provides a polypeptide including an amino acid sequence III:

MVFTLEDFVGDWRQTAGYNLDQVLEQGGVSSLFQNLGVSVTPIQRIVLS
GENGLRIDIHVIIPYEGLSGDQMGQIERIFRVVYPVDDHHFRVILHYGT
LVIDGVTPNMIDYFGRPYEGIAVFDGKKITVTGTLWNGNRIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V. The polypeptide may be selected from the group consisting of SEQ ID NOs 20 to 38.

In some embodiments, the present disclosure provides a polypeptide including an amino acid sequence IV:

MVFTLEDFVGDWRQTAGYNLDQVLEQGGVSSLFQNLGVSVTPIQRIVLS
GENGLRIDIHVIIPYEGLSGDQMGQIERIFRVVYPVDDHHFKVILHYGT
LVIDGVTPNMIDYFGRPYEGIAVFDGKKITVTGTLWNGNRIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V. The polypeptide may be selected from the group consisting of SEQ ID NOs 39 to 57.

In some embodiments, the present disclosure provides a polypeptide including an amino acid sequence V: MVFTLEDFVGDWRQTAGYNLDQVLEQGGVSSLFQNLGVSVTPIQRIVLSGENGLRID IHVIIPYEGLSGDQMGQIERIFRVVYPVDDHHFKVILHYGTLVIDGVTPNMIDYFGRPY EGIAVFDGRRITVTGTLWNGNRIIDERLINPDGSLLFRVTINGVTGWRLAERILAX₉ERILA, wherein X₉ is selected from the group consisting of A, R, N, D, E, Q, G, H, I, L, K, M, F, P, S, T, W, Y and V. The polypeptide may be selected from the group consisting of SEQ ID NOs 58 to 76.

In some embodiments, the present disclosure provides a polypeptide including an amino acid sequence VI:

MVFTLEDFVGDWRQTAGYNLDQVLEQGGVSSLFQNLGVSVTPIQKIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V. The polypeptide may be selected from the group consisting of SEQ ID NOs 77 to 95.

In some embodiments, the present disclosure provides a polypeptide including an amino acid sequence VII:

MVFTLEDFVGDWRQTAGYNLDQVLEQGGVSSLFQNLGVSVTPIQKIVLS
GENGLRIDIHVIIPYEGLSGDQMGQIERIFRVVYPVDDHHFKVILHYGT
LVIDGVTPNMIDYFGRPYEGIAVFDGRRITVTGTLWNGNRIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V. The polypeptide may be selected from the group consisting of SEQ ID NOs 96 to 114.

In some embodiments, the present disclosure provides a polypeptide including a sequence selected from the group consisting of SEQ ID NOs 1 to 114.

In some embodiments, the present disclosure provides a polynucleotide encoding any of the polypeptides described herein.

In some embodiments, the present disclosure provides a kit for luminescence assays including any of the polypeptides described herein. The kit may further comprise a substrate for the polypeptide. The substrate may be selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

In some embodiments, the present disclosure provides a method of producing luminescence including contacting any of the polypeptides described herein with a substrate for the polypeptide. The substrate may be selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

### DETAILED DESCRIPTION

Luciferases are a group of oxidative enzymes or oxygenases that catalyze a luminescent reaction that emit light. Luciferases are found in nature, for example, in bacteria (Vibrio harveyi), dinoflagellates (Gonycanulax), and the firefly (Photinus pyralis). These luciferases, in particular the eukaryotic firefly luciferase (Luc), have been commonly used as a light probe in a number of biological experiments and assays.

The term “luciferase”, as used herein, refers to an enzyme or photoprotein that catalyzes a reaction that produces bioluminescence in the presence a substrate. The term “luciferase” includes naturally occurring, recombinant, and mutant luciferases. Non-limiting examples of naturally occurring luciferases include firefly luciferase, click beetle luciferase and Renilla luciferase. Modified luciferases have also been developed. The terms modified luciferases, modified luciferase enzymes, synthetic bioluminescent luciferase proteins and synthetic luciferase polypeptides are used interchangeably herein. One example of modified luciferase is Nanoluciferase (also referred to as nanoluc, NanoLuc or Nanoluc), which is a 19.1 kDa modified luciferase polypeptide that interacts with the substrate to produce bioluminescence. The sequence for Nanoluciferase is

(SEQ ID NO: 115)
MVFTLEDFVGDWRQTAGYNLDQVLEQGGVSSLFQNLGVSVTPIQRI
VLSGENGLKIDIHVIIPYEGLSGDQMGQIEKIFKVVYPVDDHHFKVIL
HYGTLVIDGVTPNMIDYFGRPYEGIAVFDGKKITVTGTLWNGNKIID
ERLINPDGSLLFRVTINGVTGWRLCERILA.

The amino acid numbering used throughout this application to identify substituted residues is specified relative to the positions in the nanoluciferase polypeptide sequence of SEQ ID NO: 115.

Generally, luciferase generates light by the oxidation of a substrate (e.g., luciferin) that is enzyme-specific. For firefly luciferase and all other beetle luciferases, light generation occurs in the presence of magnesium ions, oxygen, and/or ATP. For Renilla luciferase, luciferin and oxygen are required.

The term “substrate” as used herein can be any luciferase substrate known in the art. Non limiting examples of substrates include furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

### I. Compositions

The present disclosure provides compounds such as polypeptides and/or proteins that produce bioluminescence and that enable more robust screening of when they are fused to a target of interest. These polypeptides show improved activities than natural luciferases and/or nanoluciferase in the presence of increasing concentrations of harsh reactant molecules. These polypeptides may have enhanced luminescence and/or enhanced protein stability compared with natural luciferases and/or nanoluciferase.

In some embodiments, the present disclosure provides polypeptides that are modified luciferase polypeptides.

The term “luminescence” as used herein, refers to the light output of the luciferase polypeptide under appropriate conditions, e.g. in the presence of a suitable substrate. The light output may be measured as a measure of light output upon start of the luminescence reaction, which may start upon addition of the substrate. In some embodiments, the luminescence reaction is carried out in a solution containing lysate. In other embodiments, expression occurs in an in vitro system. In some embodiments, the reaction is started by injecting appropriate materials, e.g. a substrate, into a reaction chamber (e.g. a well of a multiwell plate such as a 96-well plate) containing the luciferase polypeptide. The reaction chamber may be situated in a reading device which can measure the light output. The light output or luminescence may also be measured over time, for example in the same reaction chamber for a period of seconds, minutes, hours, etc. The light output or luminescence may be reported as the average over time, the half-life of decay of signal, the sum of the signal over a period of time, or as the peak output.

“Enhanced luminescence” as used herein refers to increased light output or luminescence, determined by suitable comparison with nanoluciferase measurements. In some embodiments, the modified luciferase polypeptides described herein exhibit enhanced luminescence.

“Enhanced protein stability” as used herein refers to increased thermal stability, for example stability at elevated temperatures and/or chemical stability, for example stability in the presence of harsh chemicals, such as but not limited to, urea, iodoacetamide, DMSO, formamidine, sodium dodecyl sulfate, glycerol, and Triton X-100. In some embodiments, the modified luciferase polypeptides described herein exhibit enhanced stability.

It is an object of the disclosure to provide polypeptide compositions such as synthetic luciferase polypeptides, and methods of using these polypeptide compositions.

It is also an object of the disclosure to provide a kit for luminescence assays comprising the polypeptide. In some embodiments, the kit may comprise any of the polypeptides disclosed herein. In some embodiments, the kit may further comprise a substrate for the polypeptide. In some embodiments, the substrate may be selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

In some embodiments, the compounds disclosed herein are polypeptides. According to the present disclosure, any amino acid-based molecule (natural or unnatural) may be termed a “polypeptide” and this term embraces “peptides,” “peptidomimetics,” and “proteins.” “Peptides” are traditionally considered to range in size from about 4 to about 200 amino acids. Polypeptides larger than about 200 amino acids are generally termed “proteins.” Peptides may be formed by condensation or coupling reaction with the amino group of one α-carbon carboxyl group and another amino acid. The polypeptides may be linear or cyclic. Cyclic polypeptides include any polypeptides that have as part of their structure one or more cyclic features such as a loop and/or an internal linkage.

In some embodiments, the polypeptides disclosed herein may be peptidomimetics. A “peptidomimetic” or “polypeptide mimetic” is a polypeptide in which the molecule contains structural elements that are not found in natural polypeptides (i.e., polypeptides comprised of only the 20 proteinogenic amino acids). In some embodiments, peptidomimetics are capable of recapitulating or mimicking the biological action(s) of a natural peptide. A peptidomimetic may differ in many ways from natural polypeptides, for example through changes in backbone structure or through the presence of amino acids that do not occur in nature. In some cases, peptidomimetics may include amino acids with side chains that are not found among the known 20 proteinogenic amino acids; non-polypeptide-based bridging moieties used to effect cyclization between the ends or internal portions of the molecule; substitutions of the amide bond hydrogen moiety by methyl groups (N-methylation) or other alkyl groups; replacement of a peptide bond with a chemical group or bond that is resistant to chemical or enzymatic treatments; N- and C-terminal modifications; and/or conjugation with a non-peptidic extension (such as polyethylene glycol, lipids, carbohydrates, nucleosides, nucleotides, nucleoside bases, various small molecules, or phosphate or sulfate groups).

As used herein, the term “amino acid” includes the residues of the natural amino acids as well as unnatural amino acids. The 20 natural proteinogenic amino acids are identified and referred to herein by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g. acetyl or benzyloxycarbonyl), as well as natural and unnatural amino acids protected at the carboxy terminus (e.g., as a (C1-C6) alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc., and documents cited therein, the contents of each of which are herein incorporated by reference in their entirety). Polypeptides and/or polypeptide compositions of the present disclosure may also include modified amino acids.

“Unnatural” amino acids have side chains or other features not present in the 20 naturally-occurring amino acids listed above and include, but are not limited to: N-methyl amino acids, N-alkyl amino acids, alpha, alpha substituted amino acids, beta-amino acids, alpha-hydroxy amino acids, D-amino acids, and other unnatural amino acids known in the art (See, e.g., Josephson et al., (2005) J. Am. Chem. Soc. 127: 11727-11735; Forster, A. C. et al. (2003) Proc. Natl. Acad. Sci. USA 100: 6353-6357; Subtelny et al., (2008) J. Am. Chem. Soc. 130: 6131-6136; Hartman, M. C. T. et al. (2007) PLoS ONE 2:e972; and Hartman et al., (2006) Proc. Natl. Acad. Sci. USA 103:4356-4361).

Modified amino acid residues useful for the optimization of polypeptides and/or polypeptide compositions of the present disclosure include, but are not limited to those which are chemically blocked (reversibly or irreversibly); chemically modified on their N-terminal amino group or their side chain groups; chemically modified in the amide backbone, as for example, N-methylated, D (unnatural amino acids) and L (natural amino acids) stereoisomers; or residues wherein the side chain functional groups are chemically modified to another functional group.

The present disclosure contemplates variants and derivatives of polypeptides presented herein. These include substitutional, insertional, deletional, and covalent variants and derivatives. As used herein, the term “derivative” is used synonymously with the term “variant” and refers to a molecule that has been modified or changed in any way relative to a reference molecule or starting molecule.

Modified Luciferase Polypeptides

In some embodiments, the polypeptide may comprise an amino acid sequence I:

MVFTLEDFVGDWRQTAGYNLDQVLEQGGVSSLFQNLGVSVTPIQXIIV
LSGENGLX₂IDIHVIIPYEGLSGDQMGQIEX₃IFX₄VVYPVDDHHFKVI
LHYGTLVIDGVTPNMIDYFGRPYEGIAVFDGX₅X₆ITVTGTLWNGNX₇I
IDERLINPDGSLLFRVTINGVTGWRLX₈ERILA,

wherein X₁, X₂, X₃, X₄, X₅, X₆, and X₇ are independently R or K;
X₈ is selected from the group consisting of A, R, N, D, E, Q, G, H, I, L, K, M, F, P, S, T, W, Y and V; and
optionally comprising an amino acid sequence X₉ERILA attached to rightmost A residue,
wherein X₉ is selected from the group consisting of A, R, N, D, E, Q, G, H, I, L, K, M, F, P, S, T, W, Y and V.

In some embodiments, X₁ may be R. In some embodiments, X₁ may be K.

In some embodiments, X₂ may be R. In some embodiments, X₂ may be K.

In some embodiments, X₃ may be R. In some embodiments, X₃ may be K.

In some embodiments, X₄ may be R. In some embodiments, X₄ may be K.

In some embodiments, X₅ and X₆ may both be R. In some embodiments, X₅ and X₆ may both be K.

In some embodiments, X₇ may be R. In some embodiments, X₇ may be K.

Non-limiting examples of polypeptides comprising an amino acid sequence I are shown in Table 1.

TABLE 1
Non-Limiting Examples of Polypeptides

| | SEQ ID |
|---|---|
| Sequences | NO: |
| MVFTLEDFVGDWRQTAGYNLDQVLEQGGVSSLFQNLGVSVTPIQRIVLSGENGLKI | 1 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLAERILA | |
| MVFTLEDFVGDWRQTAGYNLDQVLEQGGVSSLFQNLGVSVTPIQRIVLSGENGLKI | 2 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLRERILA | |
| MVFTLEDFVGDWRQTAGYNLDQVLEQGGVSSLFQNLGVSVTPIQRIVLSGENGLKI | 3 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLNERILA | |
| MVFTLEDFVGDWRQTAGYNLDQVLEQGGVSSLFQNLGVSVTPIQRIVLSGENGLKI | 4 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLDERILA | |
| MVFTLEDFVGDWRQTAGYNLDQVLEQGGVSSLFQNLGVSVTPIQRIVLSGENGLKI | 5 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLEERILA | |
| MVFTLEDFVGDWRQTAGYNLDQVLEQGGVSSLFQNLGVSVTPIQRIVLSGENGLKI | 6 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLQERILA | |
| MVFTLEDFVGDWRQTAGYNLDQVLEQGGVSSLFQNLGVSVTPIQRIVLSGENGLKI | 7 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLGERILA | |
| MVFTLEDFVGDWRQTAGYNLDQVLEQGGVSSLFQNLGVSVTPIQRIVLSGENGLKI | 8 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLHERILA | |
| MVFTLEDFVGDWRQTAGYNLDQVLEQGGVSSLFQNLGVSVTPIQRIVLSGENGLKI | 9 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLIERILA | |
| MVFTLEDFVGDWRQTAGYNLDQVLEQGGVSSLFQNLGVSVTPIQRIVLSGENGLKI | 10 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLLERILA | |
| MVFTLEDFVGDWRQTAGYNLDQVLEQGGVSSLFQNLGVSVTPIQRIVLSGENGLKI | 11 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLKERILA | |
| MVFTLEDFVGDWRQTAGYNLDQVLEQGGVSSLFQNLGVSVTPIQRIVLSGENGLKI | 12 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLMERILA | |
| MVFTLEDFVGDWRQTAGYNLDQVLEQGGVSSLFQNLGVSVTPIQRIVLSGENGLKI | 13 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLFERILA | |
| MVFTLEDFVGDWRQTAGYNLDQVLEQGGVSSLFQNLGVSVTPIQRIVLSGENGLKI | 14 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLPERILA | |
| MVFTLEDFVGDWRQTAGYNLDQVLEQGGVSSLFQNLGVSVTPIQRIVLSGENGLKI | 15 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLSERILA | |
| MVFTLEDFVGDWRQTAGYNLDQVLEQGGVSSLFQNLGVSVTPIQRIVLSGENGLKI | 16 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLTERILA | |
| MVFTLEDFVGDWRQTAGYNLDQVLEQGGVSSLFQNLGVSVTPIQRIVLSGENGLKI | 17 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLWERILA | |
| MVFTLEDFVGDWRQTAGYNLDQVLEQGGVSSLFQNLGVSVTPIQRIVLSGENGLKI | 18 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLYERILA | |
| MVFTLEDFVGDWRQTAGYNLDQVLEQGGVSSLFQNLGVSVTPIQRIVLSGENGLKI | 19 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLVERILA | |
| MVFTLEDFVGDWRQTAGYNLDQVLEQGGVSSLFQNLGVSVTPIQRIVLSGENGLRI | 20 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLAERILA | |
| MVFTLEDFVGDWRQTAGYNLDQVLEQGGVSSLFQNLGVSVTPIQRIVLSGENGLRI | 21 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLRERILA | |
| MVFTLEDFVGDWRQTAGYNLDQVLEQGGVSSLFQNLGVSVTPIQRIVLSGENGLRI | 22 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLNERILA | |
| MVFTLEDFVGDWRQTAGYNLDQVLEQGGVSSLFQNLGVSVTPIQRIVLSGENGLRI | 23 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLDERILA | |
| MVFTLEDFVGDWRQTAGYNLDQVLEQGGVSSLFQNLGVSVTPIQRIVLSGENGLRI | 24 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLEERILA | |
| MVFTLEDFVGDWRQTAGYNLDQVLEQGGVSSLFQNLGVSVTPIQRIVLSGENGLRI | 25 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLQERILA | |
| MVFTLEDFVGDWRQTAGYNLDQVLEQGGVSSLFQNLGVSVTPIQRIVLSGENGLRI | 26 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLGERILA | |
| MVFTLEDFVGDWRQTAGYNLDQVLEQGGVSSLFQNLGVSVTPIQRIVLSGENGLRI | 27 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLHERILA | |
| MVFTLEDFVGDWRQTAGYNLDQVLEQGGVSSLFQNLGVSVTPIQRIVLSGENGLRI | 28 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLIERILA | |
| MVFTLEDFVGDWRQTAGYNLDQVLEQGGVSSLFQNLGVSVTPIQRIVLSGENGLRI | 29 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLLERILA | |
| MVFTLEDFVGDWRQTAGYNLDQVLEQGGVSSLFQNLGVSVTPIQRIVLSGENGLRI | 30 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLKERILA | |
| MVFTLEDFVGDWRQTAGYNLDQVLEQGGVSSLFQNLGVSVTPIQRIVLSGENGLRI | 31 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLMERILA | |
| MVFTLEDFVGDWRQTAGYNLDQVLEQGGVSSLFQNLGVSVTPIQRIVLSGENGLRI | 32 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLFERILA | |
| MVFTLEDFVGDWRQTAGYNLDQVLEQGGVSSLFQNLGVSVTPIQRIVLSGENGLRI | 33 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLPERILA | |
| MVFTLEDFVGDWRQTAGYNLDQVLEQGGVSSLFQNLGVSVTPIQRIVLSGENGLRI | 34 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLSERILA | |
| MVFTLEDFVGDWRQTAGYNLDQVLEQGGVSSLFQNLGVSVTPIQRIVLSGENGLRI | 35 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLTERILA | |
| MVFTLEDFVGDWRQTAGYNLDQVLEQGGVSSLFQNLGVSVTPIQRIVLSGENGLRI | 36 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLWERILA | |
| MVFTLEDFVGDWRQTAGYNLDQVLEQGGVSSLFQNLGVSVTPIQRIVLSGENGLRI | 37 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLYERILA | |
| MVFTLEDFVGDWRQTAGYNLDQVLEQGGVSSLFQNLGVSVTPIQRIVLSGENGLRI | 38 |
| DIHVIIPYEGLSGDQMGQIERIFRVVYPVDDHHFRVILHYGTLVIDGVTPNMIDYFGR | |
| PYEGIAVFDGKKITVTGTLWNGNRIIDERLINPDGSLLFRVTINGVTGWRLVERILA | |
| MVFTLEDFVGDWRQTAGYNLDQVLEQGGVSSLFQNLGVSVTPIQRIVLSGENGLRI | 39 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLAERILA | |
| MVFTLEDFVGDWRQTAGYNLDQVLEQGGVSSLFQNLGVSVTPIQRIVLSGENGLRI | 40 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLRERILA | |
| MVFTLEDFVGDWRQTAGYNLDQVLEQGGVSSLFQNLGVSVTPIQRIVLSGENGLRI | 41 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLNERILA | |
| MVFTLEDFVGDWRQTAGYNLDQVLEQGGVSSLFQNLGVSVTPIQRIVLSGENGLRI | 42 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLDERILA | |
| MVFTLEDFVGDWRQTAGYNLDQVLEQGGVSSLFQNLGVSVTPIQRIVLSGENGLRI | 43 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLEERILA | |
| MVFTLEDFVGDWRQTAGYNLDQVLEQGGVSSLFQNLGVSVTPIQRIVLSGENGLRI | 44 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLQERILA | |
| MVFTLEDFVGDWRQTAGYNLDQVLEQGGVSSLFQNLGVSVTPIQRIVLSGENGLRI | 45 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLGERILA | |
| MVFTLEDFVGDWRQTAGYNLDQVLEQGGVSSLFQNLGVSVTPIQRIVLSGENGLRI | 46 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLHERILA | |
| MVFTLEDFVGDWRQTAGYNLDQVLEQGGVSSLFQNLGVSVTPIQRIVLSGENGLRI | 47 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLIERILA | |
| MVFTLEDFVGDWRQTAGYNLDQVLEQGGVSSLFQNLGVSVTPIQRIVLSGENGLRI | 48 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLLERILA | |
| MVFTLEDFVGDWRQTAGYNLDQVLEQGGVSSLFQNLGVSVTPIQRIVLSGENGLRI | 49 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLKERILA | |
| MVFTLEDFVGDWRQTAGYNLDQVLEQGGVSSLFQNLGVSVTPIQRIVLSGENGLRI | 50 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLMERILA | |
| MVFTLEDFVGDWRQTAGYNLDQVLEQGGVSSLFQNLGVSVTPIQRIVLSGENGLRI | 51 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLFERILA | |
| MVFTLEDFVGDWRQTAGYNLDQVLEQGGVSSLFQNLGVSVTPIQRIVLSGENGLRI | 52 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLPERILA | |
| MVFTLEDFVGDWRQTAGYNLDQVLEQGGVSSLFQNLGVSVTPIQRIVLSGENGLRI | 53 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLSERILA | |
| MVFTLEDFVGDWRQTAGYNLDQVLEQGGVSSLFQNLGVSVTPIQRIVLSGENGLRI | 54 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLTERILA | |
| MVFTLEDFVGDWRQTAGYNLDQVLEQGGVSSLFQNLGVSVTPIQRIVLSGENGLRI | 55 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLWERILA | |
| MVFTLEDFVGDWRQTAGYNLDQVLEQGGVSSLFQNLGVSVTPIQRIVLSGENGLRI | 56 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLYERILA | |
| MVFTLEDFVGDWRQTAGYNLDQVLEQGGVSSLFQNLGVSVTPIQRIVLSGENGLRI | 57 |
| DIHVIIPYEGLSGDQMGQIERIFRVVYPVDDHHFKVILHYGTLVIDGVTPNMIDYFGR | |
| PYEGIAVFDGKKITVTGTLWNGNRIIDERLINPDGSLLFRVTINGVTGWRLVERILA | |
| MVFTLEDFVGDWRQTAGYNLDQVLEQGGVSSLFQNLGVSVTPIQRIVLSGENGLRI | 58 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAA | |
| ERILA | |
| MVFTLEDFVGDWRQTAGYNLDQVLEQGGVSSLFQNLGVSVTPIQRIVLSGENGLRI | 59 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAR | |
| ERILA | |
| MVFTLEDFVGDWRQTAGYNLDQVLEQGGVSSLFQNLGVSVTPIQRIVLSGENGLRI | 60 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAN | |
| ERILA | |
| MVFTLEDFVGDWRQTAGYNLDQVLEQGGVSSLFQNLGVSVTPIQRIVLSGENGLRI | 61 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAD | |
| ERILA | |
| MVFTLEDFVGDWRQTAGYNLDQVLEQGGVSSLFQNLGVSVTPIQRIVLSGENGLRI | 62 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAE | |
| ERILA | |
| MVFTLEDFVGDWRQTAGYNLDQVLEQGGVSSLFQNLGVSVTPIQRIVLSGENGLRI | 63 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAQ | |
| ERILA | |
| MVFTLEDFVGDWRQTAGYNLDQVLEQGGVSSLFQNLGVSVTPIQRIVLSGENGLRI | 64 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAG | |
| ERILA | |
| MVFTLEDFVGDWRQTAGYNLDQVLEQGGVSSLFQNLGVSVTPIQRIVLSGENGLRI | 65 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAH | |
| ERILA | |
| MVFTLEDFVGDWRQTAGYNLDQVLEQGGVSSLFQNLGVSVTPIQRIVLSGENGLRI | 66 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAIE | |
| RILA | |
| MVFTLEDFVGDWRQTAGYNLDQVLEQGGVSSLFQNLGVSVTPIQRIVLSGENGLRI | 67 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAL | |
| ERILA | |
| MVFTLEDFVGDWRQTAGYNLDQVLEQGGVSSLFQNLGVSVTPIQRIVLSGENGLRI | 68 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAK | |
| ERILA | |
| MVFTLEDFVGDWRQTAGYNLDQVLEQGGVSSLFQNLGVSVTPIQRIVLSGENGLRI | 69 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAM | |
| ERILA | |
| MVFTLEDFVGDWRQTAGYNLDQVLEQGGVSSLFQNLGVSVTPIQRIVLSGENGLRI | 70 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAF | |
| ERILA | |
| MVFTLEDFVGDWRQTAGYNLDQVLEQGGVSSLFQNLGVSVTPIQRIVLSGENGLRI | 71 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAP | |
| ERILA | |
| MVFTLEDFVGDWRQTAGYNLDQVLEQGGVSSLFQNLGVSVTPIQRIVLSGENGLRI | 72 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAS | |
| ERILA | |
| MVFTLEDFVGDWRQTAGYNLDQVLEQGGVSSLFQNLGVSVTPIQRIVLSGENGLRI | 73 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAT | |
| ERILA | |
| MVFTLEDFVGDWRQTAGYNLDQVLEQGGVSSLFQNLGVSVTPIQRIVLSGENGLRI | 74 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAW | |
| ERILA | |
| MVFTLEDFVGDWRQTAGYNLDQVLEQGGVSSLFQNLGVSVTPIQRIVLSGENGLRI | 75 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAY | |
| ERILA | |
| MVFTLEDFVGDWRQTAGYNLDQVLEQGGVSSLFQNLGVSVTPIQRIVLSGENGLRI | 76 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILAV | |
| ERILA | |
| MVFTLEDFVGDWRQTAGYNLDQVLEQGGVSSLFQNLGVSVTPIQKIVLSGENGLKI | 77 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLAERILA | |
| MVFTLEDFVGDWRQTAGYNLDQVLEQGGVSSLFQNLGVSVTPIQKIVLSGENGLKI | 78 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLRERILA | |
| MVFTLEDFVGDWRQTAGYNLDQVLEQGGVSSLFQNLGVSVTPIQKIVLSGENGLKI | 79 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLNERILA | |
| MVFTLEDFVGDWRQTAGYNLDQVLEQGGVSSLFQNLGVSVTPIQKIVLSGENGLKI | 80 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLDERILA | |
| MVFTLEDFVGDWRQTAGYNLDQVLEQGGVSSLFQNLGVSVTPIQKIVLSGENGLKI | 81 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLEERILA | |
| MVFTLEDFVGDWRQTAGYNLDQVLEQGGVSSLFQNLGVSVTPIQKIVLSGENGLKI | 82 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLQERILA | |
| MVFTLEDFVGDWRQTAGYNLDQVLEQGGVSSLFQNLGVSVTPIQKIVLSGENGLKI | 83 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLGERILA | |
| MVFTLEDFVGDWRQTAGYNLDQVLEQGGVSSLFQNLGVSVTPIQKIVLSGENGLKI | 84 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLHERILA | |
| MVFTLEDFVGDWRQTAGYNLDQVLEQGGVSSLFQNLGVSVTPIQKIVLSGENGLKI | 85 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLIERILA | |
| MVFTLEDFVGDWRQTAGYNLDQVLEQGGVSSLFQNLGVSVTPIQKIVLSGENGLKI | 86 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLLERILA | |
| MVFTLEDFVGDWRQTAGYNLDQVLEQGGVSSLFQNLGVSVTPIQKIVLSGENGLKI | 87 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLKERILA | |
| MVFTLEDFVGDWRQTAGYNLDQVLEQGGVSSLFQNLGVSVTPIQKIVLSGENGLKI | 88 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLMERILA | |
| MVFTLEDFVGDWRQTAGYNLDQVLEQGGVSSLFQNLGVSVTPIQKIVLSGENGLKI | 89 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLFERILA | |
| MVFTLEDFVGDWRQTAGYNLDQVLEQGGVSSLFQNLGVSVTPIQKIVLSGENGLKI | 90 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLPERILA | |
| MVFTLEDFVGDWRQTAGYNLDQVLEQGGVSSLFQNLGVSVTPIQKIVLSGENGLKI | 91 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLSERILA | |
| MVFTLEDFVGDWRQTAGYNLDQVLEQGGVSSLFQNLGVSVTPIQKIVLSGENGLKI | 92 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLTERILA | |
| MVFTLEDFVGDWRQTAGYNLDQVLEQGGVSSLFQNLGVSVTPIQKIVLSGENGLKI | 93 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLWERILA | |
| MVFTLEDFVGDWRQTAGYNLDQVLEQGGVSSLFQNLGVSVTPIQKIVLSGENGLKI | 94 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLYERILA | |
| MVFTLEDFVGDWRQTAGYNLDQVLEQGGVSSLFQNLGVSVTPIQKIVLSGENGLKI | 95 |
| DIHVIIPYEGLSGDQMGQIEKIFKVVYPVDDHHFKVILHYGTLVIDGVTPNMIDYFGR | |
| PYEGIAVFDGKKITVTGTLWNGNKIIDERLINPDGSLLFRVTINGVTGWRLVERILA | |
| MVFTLEDFVGDWRQTAGYNLDQVLEQGGVSSLFQNLGVSVTPIQKIVLSGENGLRI | 96 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLAERILA | |
| MVFTLEDFVGDWRQTAGYNLDQVLEQGGVSSLFQNLGVSVTPIQKIVLSGENGLRI | 97 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLRERILA | |
| MVFTLEDFVGDWRQTAGYNLDQVLEQGGVSSLFQNLGVSVTPIQKIVLSGENGLRI | 98 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLNERILA | |
| MVFTLEDFVGDWRQTAGYNLDQVLEQGGVSSLFQNLGVSVTPIQKIVLSGENGLRI | 99 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLDERILA | |
| MVFTLEDFVGDWRQTAGYNLDQVLEQGGVSSLFQNLGVSVTPIQKIVLSGENGLRI | 100 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLEERILA | |
| MVFTLEDFVGDWRQTAGYNLDQVLEQGGVSSLFQNLGVSVTPIQKIVLSGENGLRI | 101 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLQERILA | |
| MVFTLEDFVGDWRQTAGYNLDQVLEQGGVSSLFQNLGVSVTPIQKIVLSGENGLRI | 102 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLGERILA | |
| MVFTLEDFVGDWRQTAGYNLDQVLEQGGVSSLFQNLGVSVTPIQKIVLSGENGLRI | 103 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLHERILA | |
| MVFTLEDFVGDWRQTAGYNLDQVLEQGGVSSLFQNLGVSVTPIQKIVLSGENGLRI | 104 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLIERILA | |
| MVFTLEDFVGDWRQTAGYNLDQVLEQGGVSSLFQNLGVSVTPIQKIVLSGENGLRI | 105 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLLERILA | |
| MVFTLEDFVGDWRQTAGYNLDQVLEQGGVSSLFQNLGVSVTPIQKIVLSGENGLRI | 106 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLKERILA | |
| MVFTLEDFVGDWRQTAGYNLDQVLEQGGVSSLFQNLGVSVTPIQKIVLSGENGLRI | 107 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLMERILA | |
| MVFTLEDFVGDWRQTAGYNLDQVLEQGGVSSLFQNLGVSVTPIQKIVLSGENGLRI | 108 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLFERILA | |
| MVFTLEDFVGDWRQTAGYNLDQVLEQGGVSSLFQNLGVSVTPIQKIVLSGENGLRI | 109 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLPERILA | |
| MVFTLEDFVGDWRQTAGYNLDQVLEQGGVSSLFQNLGVSVTPIQKIVLSGENGLRI | 110 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLSERILA | |
| MVFTLEDFVGDWRQTAGYNLDQVLEQGGVSSLFQNLGVSVTPIQKIVLSGENGLRI | 111 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLTERILA | |
| MVFTLEDFVGDWRQTAGYNLDQVLEQGGVSSLFQNLGVSVTPIQKIVLSGENGLRI | 112 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLWERILA | |
| MVFTLEDFVGDWRQTAGYNLDQVLEQGGVSSLFQNLGVSVTPIQKIVLSGENGLRI | 113 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLYERILA | |
| MVFTLEDFVGDWRQTAGYNLDQVLEQGGVSSLFQNLGVSVTPIQKIVLSGENGLRI | 114 |
| DIHVIIPYEGLSGDQMGQIERIFRVVYPVDDHHFKVILHYGTLVIDGVTPNMIDYFGR | |
| PYEGIAVFDGRRITVTGTLWNGNRIIDERLINPDGSLLFRVTINGVTGWRLVERILA | |

In some embodiments, the polypeptide may comprise an amino acid sequence II:

MVFTLEDFVGDWRQTAGYNLDQVLEQGGVSSLFQNLGVSVTPIQRIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V.

Non-limiting examples of polypeptides comprising an amino acid sequence II include SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

In some embodiments, the polypeptide is selected from the group consisting of SEQ ID NO: 6, SEQ ID NO: 8, SEQ ID NO: 9, SEQ ID NO: 10, SEQ ID NO: 13, SEQ ID NO: 15, SEQ ID NO: 16, SEQ ID NO: 17, SEQ ID NO: 18 and SEQ ID NO: 19.

In some embodiments, the polypeptide is selected from the group consisting of SEQ ID NO: 8, SEQ ID NO: 10, SEQ ID NO: 16, SEQ ID NO: 17, SEQ ID NO: 18 and SEQ ID NO: 19.

In some embodiments, the polypeptide comprises SEQ ID NO: 8. In some embodiments, the polypeptide comprises SEQ ID NO: 10. In some embodiments, the polypeptide comprises SEQ ID NO: 16. In some embodiments, the polypeptide comprises SEQ ID NO: 17. In some embodiments, the polypeptide comprises SEQ ID NO: 18. In some embodiments, the polypeptide comprises SEQ ID NO: 19.

In some embodiments, the polypeptide may comprise an amino acid sequence III:

MVFTLEDFVGDWRQTAGYNLDQVLEQGGVSSLFQNLGVSVTPIQRIVLS
GENGLRIDIHVIIPYEGLSGDQMGQIERIFRVVYPVDDHHFRVILHYGT
LVIDGVTPNMIDYFGRPYEGIAVFDGKKITVTGTLWNGNRIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V.

Non-limiting examples of polypeptides comprising an amino acid sequence III include SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38.

MVFTLEDFVGDWRQTAGYNLDQVLEQGGVSSLFQNLGVSVTPIQRIVLS
GENGLRIDIHVIIPYEGLSGDQMGQIERIFRVVYPVDDHHFKVILHYGT
LVIDGVTPNMIDYFGRPYEGIAVFDGKKITVTGTLWNGNRIIDERLINP
DGSLLFRVTINGVTGWRLX₈ERILA,

In some embodiments, the polypeptide may comprise an amino acid sequence IV: MVFTLEDFVGDWRQTAGYNLDQVLEQGGVSSLFQNLGVSVTPIQRIVLSGENGLRID IHVIIPYEGLSGDQMGQIERIFRVVYPVDDHHFKVILHYGTLVIDGVTPNMIDYFGRPY EGIAVFDGKKITVTGTLWNGNRIIDERLINPDGSLLFRVTINGVTGWRLX₈ERILA, wherein X₈ is selected from the group consisting of A, R, N, D, E, Q, G, H, I, L, K, M, F, P, S, T, W, Y and V.

Non-limiting examples of polypeptides comprising an amino acid sequence IV include SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56 and SEQ ID NO: 57.

In some embodiments, the polypeptide may comprise an amino acid sequence V: MVFTLEDFVGDWRQTAGYNLDQVLEQGGVSSLFQNLGVSVTPIQRIVLSGENGLRID IHVIIPYEGLSGDQMGQIERIFRVVYPVDDHHFKVILHYGTLVIDGVTPNMIDYFGRPY EGIAVFDGRRITVTGTLWNGNRIIDERLINPDGSLLFRVTINGVTGWRLAERILAX₉ERI LA, wherein X₉ is selected from the group consisting of A, R, N, D, E, Q, G, H, I, L, K, M, F, P, S, T, W, Y and V.

Non-limiting examples of polypeptides comprising an amino acid sequence V include SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76.

In some embodiments, the polypeptide may comprise an amino acid sequence VI:

MVFTLEDFVGDWRQTAGYNLDQVLEQGGVSSLFQNLGVSVTPIQKIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V.

Non-limiting examples of polypeptides comprising an amino acid sequence VI include SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94 and SEQ ID NO: 95.

In some embodiments, the polypeptide may comprise an amino acid sequence VII:

MVFTLEDFVGDWRQTAGYNLDQVLEQGGVSSLFQNLGVSVTPIQKIVLS
GENGLRIDIHVIIPYEGLSGDQMGQIERIFRVVYPVDDHHFKVILHYGT
LVIDGVTPNMIDYFGRPYEGIAVFDGRRITVTGTLWNGNRIIDERLINP
DGSLLFRVTINGVTGWRLX₈ERILA,

wherein X₈ is selected from the group consisting of A, R, N, D, E, Q, G, H, I, L, K, M, F, P, S, T, W, Y and V.

Non-limiting examples of polypeptides comprising an amino acid sequence VII include SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113 and SEQ ID NO: 114.

In some embodiments, the polypeptide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113 and SEQ ID NO: 114.

In some embodiments, the polypeptide may be fused to a biological target to form a fusion protein. In some embodiments, the polypeptide may not be fused to a biological target. In some embodiments, the biological target is a protein, polynucleotide, or lipid.

The polypeptides of the present disclosure and/or the fusion proteins comprising the polypeptides may be prepared with any method known in the art. The modified luciferase polypeptides or fusion proteins of the disclosure may be prepared by recombinant methods or by solid phase chemical peptide synthesis methods. In some cases, the polypeptides and/or the fusion proteins of the present disclosure can be prepared from nucleic acid molecules encoding the polypeptides.

The terms “nucleic acid molecule”, “polynucleotide” and “nucleic acid sequence” as used herein, refer to nucleic acid including DNA or RNA that comprises coding sequences necessary for the production of a polypeptide or protein precursor. The nucleic acid molecules encoding the modified luciferase polypeptides and/or the fusion proteins of the present disclosure can be designed by methods known in the art.

Nucleic acids are known to contain different types of mutations, which refers to an alteration in the sequence of a nucleotide at a particular base position relative to the wild-type sequence. Mutations may also refer to insertion or deletion of one or more bases so that the nucleic acid sequence differs from a reference. The term “substitution” as used herein refers to a change in an amino acid at a particular position in a sequence. The term “vector” as used herein refers to nucleic acid molecules into which fragments of DNA may be inserted or cloned and can be used to transfer DNA segments into a cell and capable of replication in a cell.

In some embodiments, the polynucleotides disclosed herein may encode the polypeptides comprising a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113 and SEQ ID NO: 114.

### II. Methods of Using the Polypeptides

The polypeptides of the present disclosure can be used in any way that luciferases and luciferase substrates have been used in the art. For example, the polypeptides may be used in a bioluminogenic assay to detect one or more molecules in a sample, e.g., an enzyme, a cofactor for an enzymatic reaction, an enzyme substrate, an enzyme inhibitor, an enzyme activator, or OH radicals, or one or more conditions, e.g., redox conditions. The sample may include an animal, a plant, a fungus, physiological fluid, such as but not limited to blood, plasma, urine, mucous secretions, a cell, a cell lysate, a cell supernatant, or a purified fraction of a cell. In some embodiments, the polypeptides of the present disclosure can be used to identify drug compounds. In some embodiments, the drug compounds can be small molecules. The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

In some embodiments, the polypeptides of the present disclosure can be used to transfer energy to an energy acceptor, for example in Bioluminescence Resonance Energy Transfer (BRET) analysis. In some embodiments, the polypeptides of the present disclosure in combination with luciferase substrates can be used for detecting luminescence. In some embodiments, the substrate is selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

The methods described herein can be performed by utilizing any of a wide range cell assay formats, including, but not limited to cell plates, e.g., 24-well plates, 48-well plates, 96-well plates, or 384-well plates, individual cell culture plates, or flasks, for example T-flasks or shaker flasks.

Examples of human cell lines useful in methods provided herein as target cells include, but are not limited to, 293T (embryonic kidney), 786-0 (renal), A498 (renal), A549 (alveolar basal epithelial), ACHN (renal), BT-549 (breast), BxPC-3 (pancreatic), CAKI-1 (renal), Capan-1 (pancreatic), CCRF-CEM (leukemia), COLO 205 (colon), DLD-1 (colon), DMS 114 (small cell lung), DU145 (prostate), EKVX (non-small cell lung), HCC-2998 (colon), HCT-15 (colon), HCT-116 (colon), HT29 (colon), HT-1080 (fibrosarcoma), HEK 293 (embryonic kidney), HeLa (cervical carcinoma), HepG2 (hepatocellular carcinoma), HL-60(TB) (leukemia), HOP-62 (non-small cell lung), HOP-92 (non-small cell lung), HS 578T (breast), HT-29 (colon adenocarcinoma), IGR-OV1 (ovarian), IMR32 (neuroblastoma), Jurkat (T lymphocyte), K-562 (leukemia), KM12 (colon), KM20L2 (colon), LAN5 (neuroblastoma), LNCap.FGC (Caucasian prostate adenocarcinoma), LOX IMVI (melanoma), LXFL 529 (non-small cell lung), M14 (melanoma), M19-MEL (melanoma), MALME-3M (melanoma), MCFlOA (mammary epithelial), MCF7 (mammary), MDA-MB-453 (mammary epithelial), MDA-MB-468 (breast), MDA-MB-231 (breast), MDA-N(breast), MOLT-4 (leukemia), NCI/ADR-RES (ovarian), NCI-H226 (non-small cell lung), NCI-H23 (non-small cell lung), NCI-H322M (non-small cell lung), NCI-H460 (non-small cell lung), NCI-H522 (non-small cell lung), OVCAR-3 (ovarian), OVCAR-4 (ovarian), OVCAR-5 (ovarian), OVCAR-8 (ovarian), P388 (leukemia), P388/ADR (leukemia), PC-3 (prostate), PERC6® (El-transformed embryonal retina), RPMI-7951 (melanoma), RPMI-8226 (leukemia), RXF 393 (renal), RXF-631 (renal), Saos-2 (bone), SF-268 (CNS), SF-295 (CNS), SF-539 (CNS), SHP-77 (small cell lung), SH-SY5Y (neuroblastoma), SK-BR3 (breast), SK-MEL-2 (melanoma), SK-MEL-5 (melanoma), SK-MEL-28 (melanoma), SK-OV-3 (ovarian), SN12K1 (renal), SN12C (renal), SNB-19 (CNS), SNB-75 (CNS) SNB-78 (CNS), SR (leukemia), SW-620 (colon), T-47D (breast), THP-1 (monocyte-derived macrophages), TK-10 (renal), U87 (glioblastoma), U293 (kidney), U251 (CNS), UACC-257 (melanoma), UACC-62 (melanoma), UO-31 (renal), W138 (lung), and XF 498 (CNS).

Examples of rodent cell lines useful in methods provided herein include, but are not limited to, baby hamster kidney (BHK) cells (e.g., BHK21 cells, BHK TK− cells), mouse Sertoli (TM4) cells, buffalo rat liver (BRL 3A) cells, mouse mammary tumor (MMT) cells, rat hepatoma (HTC) cells, mouse myeloma (NS0) cells, murine hybridoma (Sp2/0) cells, mouse thymoma (EL4) cells, Chinese Hamster Ovary (CHO) cells and CHO cell derivatives, murine embryonic (NIH/3T3, 3T3 L1) cells, rat myocardial (H9c2) cells, mouse myoblast (C2C12) cells, and mouse kidney (miMCD-3) cells.

Examples of non-human primate cell lines useful in methods provided herein include, but are not limited to, monkey kidney (CVI-76) cells, African green monkey kidney (VERO-76) cells, green monkey fibroblast (Cos-1) cells, and monkey kidney (CVI) cells transformed by SV40 (Cos-7). Additional mammalian cell lines are known to those of ordinary skill in the art and are catalogued at the American Type Culture Collection catalog (ATCC®, Manassas, VA).

In some embodiments, the cells are lysed using chemical and/or mechanical lysis. In some embodiments, the chemical lysis comprises a lysis buffer comprising a protease inhibitor, phosphate buffered saline and Triton X₁₀₀. In some embodiments, the cells can be frozen after the addition of the lysis buffer at −80° C. for about 30 minutes to about 72 hours. Alternatively, the cell lysate may be stored in a range of 2 to 8° C. or at room temperature. In some embodiments, the cells are centrifuged, and cell lysates are collected. In some embodiments, this is performed by spinning the cells in a centrifuge at 3,750 RPM for 10 minutes at room temperature.

In some embodiments, the polypeptide may be fused to a biological target to form a fusion protein. In some embodiments, the biological target is a protein. In some cases, the binding of a molecule (such as an inhibitor) to the biological target changes the luminescence emitted from the polypeptide. Therefore, the luminescence emitted from the polypeptide can be used to evaluate the bindings of molecules (such as inhibitors) to the biological target.

### III. Kits and Devices

The present disclosure provides a variety of kits for conveniently and/or effectively carrying out methods of the present disclosure. Typically, kits will comprise sufficient amounts and/or numbers of components to allow a user to perform multiple experiments.

In one embodiment, the present disclosure provides kits for detecting luminescence, comprising the polypeptides of the present disclosure. In some embodiments, the kit may comprise luciferase substrates. Luciferase substrates include, but are not limited to furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the polypeptides in the buffer solution over a period of time and/or under a variety of conditions.

The present disclosure provides for devices which may incorporate the polypeptides of the present disclosure. Non-limiting examples of the devices include a pump, a needle, iontophoresis devices, multi-layered microfluidic devices.

### A. Assays

Luminescence for the polypeptides disclosed herein may be determined using various assays known in the art. As used herein, the term “assay” refers to the sequence of activities associated with a reported result, which can include, but is not limited to: cell seeding, preparation of the test material, infection, lysis, analysis, and calculation of results.

In some embodiments, the assay surfaces are sterile and are suitable for culturing cells under conditions representative of the culture conditions during large-scale (e.g., industrial scale) production of the biological product. In some embodiments, the exterior comprises wells, indentations, demarcations, or the like at positions corresponding to the assay surfaces. In some embodiments, the wells, indentations, demarcations, or the like retain fluid, such as cell culture media, over the assay surfaces.

In some embodiments, the material comprises a microarray plate, a biochip, or the like which allows for the high-throughput, automated testing of a range of test agents, conditions, and/or combinations thereof on the production of a biological product by cultured cells. For example, the material may comprise a 2-dimensional microarray plate or biochip having m columns and n rows of assay surfaces (e.g., residing within wells) which allow for the testing of m×n combinations of test agents and/or conditions (e.g., on a 24-, 96- or 384-well microarray plate). The microarray materials are preferably designed such that all necessary positive and negative controls can be carried out in parallel with testing of the agents and/or conditions.

### IV. Definitions

As used herein, the term “polypeptide” embraces “peptides,” “peptidomimetics,” and “proteins” refers to any amino acid-based molecule (natural or unnatural). “Peptides” are traditionally considered to range in size from about 4 to about 200 amino acids. Polypeptides larger than about 200 amino acids are generally termed “proteins.” Peptides may be formed by condensation or coupling reaction with the amino group of one α-carbon carboxyl group and another amino acid. The polypeptides may be linear or cyclic. Cyclic polypeptides include any polypeptides that have as part of their structure one or more cyclic features such as a loop and/or an internal linkage.

As used herein, the term “amino acid” includes the residues of the natural amino acids as well as unnatural amino acids. The 20 natural proteinogenic amino acids are identified and referred to herein by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g. acetyl or benzyloxycarbonyl), as well as natural and unnatural amino acids protected at the carboxy terminus (e.g., as a (C1-C6) alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc., and documents cited therein, the contents of each of which are herein incorporated by reference in their entirety). Polypeptides and/or polypeptide compositions of the present disclosure may also include modified amino acids.

The term “luciferase”, as used herein, refers to one or more oxidative enzymes or oxygenases that catalyze a luminescent reaction. Thus, luciferase refers to an enzyme or photoprotein that catalyzes a reaction that produces bioluminescence. The luciferase of the present disclosure may be recombinant or naturally occurring luciferase, or may be a mutant thereof. Non-limiting examples of naturally occurring luciferases include firefly luciferase, click beetle luciferase and Renilla luciferase. In some embodiments, the modified luciferases are polypeptides. The terms modified luciferases, synthetic bioluminescent proteins and synthetic luciferase proteins are used interchangeably herein.

Nanoluciferase or nanoluc refers to a 19.1 kDa luciferase enzyme that interacts with the substrate to produce bioluminescence. The sequence for Nanoluciferase is MVFTLEDFVGDWRQTAGYNLDQVLEQGGVSSLFQNLGVSVTPIQRIVLSGENGLKID IHVIIPYEGLSGDQMGQIEKIFKVVYPVDDHHFKVILHYGTLVIDGVTPNMIDYFGRP YEGIAVFDGKKITVTGTLWNGNKIIDERLINPDGSLLFRVTINGVTGWRLCERILA; listed as SEQ ID NO: 115.

The terms “nucleic acid molecule”, “polynucleotide” and “nucleic acid sequence” as used herein, refer to nucleic acid including DNA or RNA that comprises coding sequences necessary for the production of a polypeptide or protein precursor. The encoded polypeptide may be a full-length polypeptide, a fragment thereof, or a fusion of either the full-length polypeptide or fragment thereof with another polypeptide, yielding a fusion polypeptide.

The term “substitution” as used herein refers to a change in an amino acid at a particular position in a sequence.

The term “vector” as used herein refers to nucleic acid molecules into which fragments of DNA may be inserted or cloned and can be used to transfer DNA segments into a cell and capable of replication in a cell.

The term “luminescence” as used herein, refers to the light output of the luciferase polypeptide under appropriate conditions, e.g. in the presence of a suitable substrate such as a coelenterazine. The substrate may be selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine. The light output may be measured as a measure of light output upon start of the luminescence reaction, which may start upon addition of the substrate. In some embodiments, the luminescence reaction is carried out in a solution containing lysate. In other embodiments, expression occurs in an in vitro system. In some embodiments, the reaction is started by injecting appropriate materials, e.g. substrate, into a reaction chamber (e.g. a well of a multiwell plate such as a 96-well plate) containing the luciferase polypeptide. The reaction chamber may be situated in a reading device which can measure the light output. The light output or luminescence may also be measured over time, for example in the same reaction chamber for a period of seconds, minutes, hours, etc. The light output or luminescence may be reported as the average over time, the half-life of decay of signal, the sum of the signal over a period of time, or as the peak output.

“Enhanced luminescence” as used herein refers to increased light output or luminescence, determined by suitable comparison with nanoluciferase measurements. In some embodiments, the modified luciferase polypeptides described herein exhibit enhanced luminescence.

“Enhanced protein stability” as used herein refers to increased thermal stability, for example stability at elevated temperatures and/or chemical stability, for example stability in the presence of urea, iodoacetamide, DMSO, formamidine, sodium dodecyl sulfate and Triton X-100. In some embodiments, the modified luciferase polypeptides described herein exhibit enhanced stability.

As used herein, the term “assay” refers to the sequence of activities associated with a reported result, which can include, but is not limited to: cell seeding, preparation of the test material, infection, lysis, analysis, and calculation of results.

The term “compound”, as used herein, is meant to include all stereoisomers, geometric isomers, tautomers, and isotopes of the structures depicted. In some embodiments, compound is used interchangeably with the polypeptide. Therefore, polypeptide, as used herein, is also meant to include all stereoisomers, geometric isomers, tautomers, and isotopes of the structures depicted.

The compounds herein described may have asymmetric centers, geometric centers (e.g., double bond), or both. All chiral, diastereomeric, racemic forms and all geometric isomeric forms of a structure are intended, unless the specific stereochemistry or isomeric form is specifically indicated. Compounds of the present invention containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the compounds described herein, and all such stable isomers are included in the present invention. Specifically, cis and trans geometric isomers of the compounds of the present invention may also exist and may be isolated as a mixture of isomers or as separated isomeric forms. All processes used to prepare compounds of the present invention and intermediates made therein are considered to be part of the present invention.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

A “target”, as used herein, shall mean a site to which polypeptides bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). A target may be a type of tissue, e.g., neuronal tissue, intestinal tissue, pancreatic tissue, liver, kidney, prostate, ovary, lung, bone marrow, or breast tissue.

The “target cells” are generally animal cells, e.g., mammalian cells. The present method may be used to modify cellular function of living cells in vitro, i.e., in cell culture, or in vivo, in which the cells form part of or otherwise exist in animal tissue. Thus, the target cells may include, for example, the blood, lymph tissue, cells lining the alimentary canal, such as the oral and pharyngeal mucosa, cells forming the villi of the small intestine, cells lining the large intestine, cells lining the respiratory system (nasal passages/lungs) of an animal (which may be contacted by inhalation of the subject), dermal/epidermal cells, cells of the vagina and rectum, cells of internal organs including cells of the placenta and the so-called blood/brain barrier, etc.

The terms “sufficient” and “effective”, as used interchangeably herein, refer to an amount (e.g., mass, volume, dosage, concentration, and/or time period) needed to achieve one or more desired result(s).

The term “prodrug” refers to an agent, including a nucleic acid or protein that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J Drug Metab. Pharmacokinet., 15(2): 143-53; Balimane and Sinko (1999). Involvement of multiple transporters in the oral absorption of nucleoside analogs, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. DrugMetab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays, or complementation, e.g., genetic, or biochemical. See, for example, Fields et al. (1989) Nature 340:245-246; U.S. Pat. No. 5,585,245 and PCT WO 98/44350.

The term “detectable response” as used herein refers to an occurrence of, or a change in, a signal that is directly or indirectly detectable either by observation or by instrumentation. Typically, the detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### EXAMPLES

### Example 1: General Synthesis of the Polypeptides

Polypeptides of the present disclosure can be synthesized by one skilled in the art using recombinant methods including expressing luciferase in human cells fused to any protein of interest. A polynucleotide encoding a protein or polypeptide as used herein refers to a nucleic acid sequence comprising the coding region of a gene. The polynucleotides are synthesized according to methods known in the art. Non-limiting examples of polynucleotide sequences are shown below. Standard sequencing techniques known in the art were used to identify the amino acid substitution in each clone of interest.

Nucleic Acid Sequences

atggtcttcacactcgaagatttcgttggggactggcgacagacagccggctacaacctggaccaagtccttgaacagggaggtgtgtccagtt
tgtttcagaatctcggggtgtccgtaactccgatccaaaggattgtcctgagcggtgaaaatgggctgaagatcgacatccatgtcatcatccc
gtatgaaggtctgagcggcgaccaaatgggccagatcgaaaaaatttttaaggtggtgtaccctgtggatgatcatcactttaaggtgatcctg
cactatggcacactggtaatcgacggggttacgccgaacatgatcgactatttcggacggccgtatgaaggcatcgccgtgttcgacggcaaaa
agatcactgtaacagggaccctgtggaacggcaacaaaattatcgacgagcgcctgatcaaccccgacggctccctgctgttccgagtaaccat
caacggagtgaccggctggcggctgtgcgaacgcattctggcgtaa (SEQ ID No: 118, encoding polypeptide SEQ ID
No: 115)
atggtcttcacactcgaagatttcgttggggactggcgacagacagccggctacaacctggaccaagtccttgaacagggaggtgtgtccagtt
tgtttcagaatctcggggtgtccgtaactccgatccaaaggattgtcctgagcggtgaaaatgggctgaagatcgacatccatgtcatcatccc
gtatgaaggtctgagcggcgaccaaatgggccagatcgaaaaaatttttaaggtggtgtaccctgtggatgatcatcactttaaggtgatcctg
cactatggcacactggtaatcgacggggttacgccgaacatgatcgactatttcggacggccgtatgaaggcatcgccgtgttcgacggcaaaa
agatcactgtaacagggaccctgtggaacggcaacaaaattatcgacgagcgcctgatcaaccccgacggctccctgctgttccgagtaaccat
caacggagtgaccggctggcggctgggcgaacgcattctggcgtaa (SEQ ID No: 119, encoding polypeptide SEQ ID
No: 1)
atggtcttcacactcgaagatttcgttggggactggcgacagacagccggctacaacctggaccaagtccttgaacagggaggtgtgtccagtt
tgtttcagaatctcggggtgtccgtaactccgatccaaaggattgtcctgagcggtgaaaatgggctgaagatcgacatccatgtcatcatccc
gtatgaaggtctgagcggcgaccaaatgggccagatcgaaaaaatttttaaggtggtgtaccctgtggatgatcatcactttaaggtgatcctg
cactatggcacactggtaatcgacggggttacgccgaacatgatcgactatttcggacggccgtatgaaggcatcgccgtgttcgacggcaaaa
agatcactgtaacagggaccctgtggaacggcaacaaaattatcgacgagcgcctgatcaaccccgacggctccctgctgttccgagtaaccat
caacggagtgaccggctggcggctgcgcgaacgcattctggcgtaa (SEQ ID No: 120, encoding polypeptide SEQ ID
No: 2)

Alternatively, the polypeptides are synthesized by solid phase chemical peptide synthesis methods. The polypeptides are constructed from their individual amino acids. The amino acids can be covalently bonded to one another through functional groups, as is known in the art, where such functional groups may be present on the amino acids or introduced onto the components using one or more steps. When necessary and/or desired, certain moieties on the amino acids may be protected using blocking groups, as is known in the art, see, e.g., Green & Wuts, Protective Groups in Organic Synthesis (John Wiley & Sons) (1991).

For example, the polypeptides are synthesized using standard solid-phase Fmoc/tBu methods. The synthesis is typically performed on a peptide synthesizer using standard protocols with Rink amide resin. All amino acids are obtained from commercial sources unless otherwise noted. Coupling reagents known in the art can be used. Generally, the coupling reagent is 2-(6-chloro-1-H-benzotriazole-1yl)-1,1,3,3,-tetramethylaminium hexafluorophosphate (HCTU) and the base is diisopropylethylamine (DIEA). Peptides are generally cleaved from resin with trifluoroacetic acid (TFA) and water. The crude peptides are then purified on HPLC. Fractions containing the pure peptide are collected and lyophilized and all peptides are analyzed by LC-MS.

### Example 2: Transfection Protocol and Luminescence Analysis

Human embryonic kidney 293-H (HEK 293, Gibco 293-H, #11631017) cell lines were maintained in Dulbecco's Modified Eagle Medium, high glucose, pyruvate (DMEM, Gibco, #11995065) supplemented with 10% fetal bovine serum (FBS, Gibco, #10082147) and 1× penicillin-streptomycin (100× solution, Gibco, #15140148) at 37° C. and 5% C02 in a water-saturated incubator. Cell were trypsinized using 0.05% or 0.25% Trypsin-EDTA solution (Trypsin-EDTA, phenol red, Gibco, #25200056 (0.25%) or #25300054).

HEK293 cells were cultivated appropriately prior to assay. The medium from cell flask was removed via aspiration, washed 1× with PBS followed by aspiration, trypsinized, and cells were allowed to dissociate from the flask. Trypsin was neutralized using growth medium and cells were pelleted via centrifugation at 200×g for 5 minutes. The medium was aspirated, and the cells were resuspended into a single cell suspension using Opti-MEM I supplemented with 10% FBS. The cell density was adjusted to 2×10⁵/mL in Opti-MEM I supplemented with 10% FBS in a sterile, conical tube. The cells were transfected and aliquoted directly in a 96-well plate for the assay the next day. The cells were also transfected in bulk and dispensed into a 96-well plate to allow cells to adhere to the plate overnight, thereby enabling washout studies.

The lipid:DNA complexes were prepared as follows:

A 10 μg/mL solution of DNA was prepared in Opti-MEM without serum. This solution contained the following ratios of carrier DNA and DNA encoding NanoLuc fused to the biological target. Serial dilution steps may be warranted to accurately dilute the NanoLuc fusion DNA. The following reagents were added to a sterile polystyrene test tube in order: 1 mL of Opti-MEM without phenol red; 9.0 μg/mL of carrier DNA; 1.0 μg/mL of NanoLuc fusion DNA (for some targets, the amount is less). The reagents were mixed thoroughly.

30 μL of FuGENE HD was added into each mL of DNA mixture to form lipid:DNA complex. Care was taken such that FuGENE HD did not touch the plastic side of the tube and pipetted directly into the liquid in the tube. It was mixed by pipetting up and down 5-10 times and incubated at room temperature for 20 minutes to allow complexes to form. 1 part (e.g. 1 mL) of lipid:DNA complex was mixed with 20 parts (e.g. 20 mL) of HEK293 cells in suspension at 2×10⁵/mL and mixed gently by pipetting up and down 5 times in a sterile, conical tube. Larger or smaller bulk transfections were scaled accordingly, using this ratio. 100 μL cells+lipid:DNA complex was dispensed into a sterile, tissue-culture treated 96-well plate (20,000 cells/well), and incubated at least 16 hours to allow expression. The cells were incubated in a 37° C.+5% CO2 incubator for >16 hrs. Thus, the polypeptides of the present disclosure were produced.

For luminescence analysis, a 1× solution of furimazine substrate mix (500× stock) was prepared. 100 μL of the 1× Substrate-Tracer solution was added and the 96 well plate was gently tapped to mix. The cells were lysed with 1×SDS buffer and increasing concentrations of harsh chemicals: urea, iodoacetamide, SDS, DMSO, and formdine, were added at the indicated concentrations. Luminescence emitted from each polypeptide was measured.

Luminescence measurements for some non-limiting examples of modified luciferase polypeptides washed by 1 μM, 10 μM, 100 μM, or 1000 μM of iodoacetamide, SDS, and urea are shown in Table 2. NanoLuc (SEQ TD NO: 115), YeLuc (SEQ ID NO: 116) and TeLuc (SEQ ID No: 117) were used as controls.

TABLE 2
Luminescence assay data

| | Luciferase Units | | | | |
|---|---|---|---|---|---|
| Sequence ID | 0 μM | 1 μM | 10 μM | 100 μM | 1000 μM |
| SEQ ID NO: 115 | 4.67E+08 | 4.64E+08 | 3.43E+08 | 3.61E+08 | 3.03E+08 |
| SEQ ID NO: 1 | 8.75E+06 | 8.40E+06 | 7.86E+06 | 8.70E+06 | 8.51E+06 |
| SEQ ID NO: 2 | 1.02E+07 | 1.08E+07 | 1.21E+07 | 1.03E+07 | 1.22E+07 |
| SEQ ID NO: 3 | 1.07E+07 | 9.29E+06 | 9.12E+06 | 1.15E+07 | 8.87E+06 |
| SEQ ID NO: 4 | 6.62E+06 | 7.48E+06 | 6.56E+06 | 6.89E+06 | 7.02E+06 |
| SEQ ID NO: 5 | 8.04E+06 | 9.09E+06 | 7.56E+06 | 9.98E+06 | 7.33E+06 |
| SEQ ID NO: 6 | 1.80E+08 | 9.34E+07 | 2.05E+08 | 1.53E+08 | 1.51E+08 |
| SEQ ID NO: 7 | 1.49E+07 | 1.29E+07 | 1.23E+07 | 1.24E+07 | 1.27E+07 |
| SEQ ID NO: 8 | 7.01E+08 | 6.31E+08 | 6.32E+08 | 5.76E+08 | 5.14E+08 |
| SEQ ID NO: 9 | 3.67E+08 | 3.04E+08 | 2.76E+08 | 2.58E+08 | 2.48E+08 |
| SEQ ID NO: 10 | 4.94E+08 | 4.42E+08 | 4.18E+08 | 3.59E+08 | 3.64E+08 |
| SEQ ID NO: 11 | 1.07E+07 | 1.07E+07 | 1.01E+07 | 9.44E+06 | 9.98E+06 |
| SEQ ID NO: 12 | 1.89E+07 | 2.16E+07 | 1.90E+07 | 2.10E+07 | 1.81E+07 |
| SEQ ID NO: 13 | 1.33E+08 | 1.52E+08 | 2.12E+08 | 2.19E+08 | 2.39E+08 |
| SEQ ID NO: 14 | 1.09E+08 | 4.54E+07 | 3.27E+07 | 4.23E+07 | 7.78E+07 |
| SEQ ID NO: 15 | 1.82E+08 | 3.02E+08 | 3.49E+08 | 2.82E+08 | 3.00E+08 |
| SEQ ID NO: 16 | 2.86E+09 | 2.35E+09 | 1.66E+09 | 1.88E+09 | 1.26E+09 |
| SEQ ID NO: 17 | 7.52E+08 | 4.68E+08 | 6.94E+08 | 4.61E+08 | 9.23E+08 |
| SEQ ID NO: 18 | 2.71E+09 | 6.40E+08 | 4.54E+08 | 6.75E+08 | 7.75E+08 |
| SEQ ID NO: 19 | 3.05E+08 | 7.56E+08 | 8.22E+08 | 8.16E+08 | 5.25E+08 |
| SEQ ID NO: 116 | 1.86E+08 | 2.80E+08 | 1.12E+08 | 2.33E+08 | 2.40E+08 |
| SEQ ID NO: 117 | 7.40E+08 | 1.21E+08 | 9.17E+07 | 9.88E+07 | 8.81E+07 |

TABLE 3
Sequences of the controls

YeLuc:
MVLTLEDFVGDWRQTDGYNQAQVLEQGGLTSLFQNLGVSVTPIQRIVLSGENGLKIDIHVIIPYEGLS
GDRMGQIEKIFKVVYPVDDHHFKVILHYGTLVIDGVTPNMIDYFGQPYEGIAVFDGKKITVTGTLWN
GNKIIDERRINPDGSLLFRVTINGVTGWRLSERILA
(SEQ ID NO: 116)
TeLuc:
MVFTLEDFVGDWRQTAGYNLSQVLEQGGVSSLFQNLGVSVTPIQRIVLSGENGLKIDIHVIIPYEGLS
GDQMGQIEKIFKVVYPVDNHHFKVILHYGTLVIDGVTPNMIDYFGRPYEGIAVFDGKKITVTGTLWN
GNKIIDERLINPDGSLLFRVTINGVTGWRLHERILA
(SEQ ID NO: 117)

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A polypeptide comprising an amino acid sequence I:
MVFTLEDFVGDWRQTAGYNLDQVLEQGGVSSLFQNLGVSVTPIQX₁IVLSGENG LX₂IDIHVIIPYEGLSGDQMGQIEX₃IFX₄VVYPVDDHHFKVILHYGTLVIDGVTPN MIDYFGRPYEGIAVFDGX₅X₆ITVTGTLWNGNX₇IIDERLINPDGSLLFRVTINGVT GWRLX₈ERILA, wherein X₁, X₂, X₃, X₄, X₅, X₆, and X₇ are independently R or K; X₈ is selected from the group consisting of A, R, N, D, E, Q, G, H, I, L, K, M, F, P, S, T, W, Y and V; and optionally comprising an amino acid sequence X₉ERILA attached to rightmost A residue, wherein X₉ is selected from the group consisting of A, R, N, D, E, Q, G, H, I, L, K, M, F, P, S, T, W, Y and V.

2. The polypeptide of claim 1, wherein X₁ is R.

3. The polypeptide of claim 1, wherein X₁ is K.

4. The polypeptide of any one of claims 1-3, wherein X₂ is R.

5. The polypeptide of any one of claims 1-3, wherein X₂ is K.

6. The polypeptide of any one of claims 1-5, wherein X₃ is R.

7. The polypeptide of any one of claims 1-5, wherein X₃ is K.

8. The polypeptide of any one of claims 1-7, wherein X₄ is R.

9. The polypeptide of any one of claims 1-7, wherein X₄ is K.

10. The polypeptide of any one of claims 1-9, wherein X₅ and X₆ are both R.

11. The polypeptide of any one of claims 1-9, wherein X₅ and X₆ are both K.

12. The polypeptide of any one of claims 1-11, wherein X₇ is R.

13. The polypeptide of any one of claims 1-11, wherein X₇ is K.

14. The polypeptide of claim 1, comprising an amino acid sequence II:
MVFTLEDFVGDWRQTAGYNLDQVLEQGGVSSLFQNLGVSVTPIQRIVLSGENGL KIDIHVIIPYEGLSGDQMGQIEKIFKVVYPVDDHHFKVILHYGTLVIDGVTPNMID YFGRPYEGIAVFDGKKITVTGTLWNGNKIIDERLINPDGSLLFRVTINGVTGWRL X₈ERILA, wherein X₈ is selected from the group consisting of A, R, N, D, E, Q, G, H, I, L, K, M, F, P, S, T, W, Y and V.

15. The polypeptide of claim 14, wherein said polypeptide is selected from the group consisting of SEQ ID NOs 1 to 19.

16. The polypeptide of claim 15, wherein said polypeptide is selected from the group consisting of SEQ ID NO: 6, SEQ ID NO: 8, SEQ ID NO: 9, SEQ ID NO: 10, SEQ ID NO: 13, SEQ ID NO: 15, SEQ ID NO: 16, SEQ ID NO: 17, SEQ ID NO: 18 and SEQ ID NO: 19.

17. The polypeptide of claims 15 or 16, wherein said polypeptide is selected from the group consisting of SEQ ID NO:8, SEQ ID NO: 10, SEQ ID NO: 16, SEQ ID NO: 17, SEQ ID NO: 18 and SEQ ID NO:19.

18. The polypeptide of claim 1, comprising an amino acid sequence III:
MVFTLEDFVGDWRQTAGYNLDQVLEQGGVSSLFQNLGVSVTPIQRIVLSGENGL RIDIHVIIPYEGLSGDQMGQIERIFRVVYPVDDHHFRVILHYGTLVIDGVTPNMID YFGRPYEGIAVFDGKKITVTGTLWNGNRIIDERLINPDGSLLFRVTINGVTGWRL X₈ERILA, wherein X₈ is selected from the group consisting of A, R, N, D, E, Q, G, H, I, L, K, M, F, P, S, T, W, Y and V.

19. The polypeptide of claim 18, wherein said polypeptide is selected from the group consisting of SEQ ID NOs 20 to 38.

20. The polypeptide of claim 1, comprising an amino acid sequence IV:
MVFTLEDFVGDWRQTAGYNLDQVLEQGGVSSLFQNLGVSVTPIQRIVLSGENGL RIDIHVIIPYEGLSGDQMGQIERIFRVVYPVDDHHFKVILHYGTLVIDGVTPNMID YFGRPYEGIAVFDGKKITVTGTLWNGNRIIDERLINPDGSLLFRVTINGVTGWRL X₈ERILA, wherein X₈ is selected from the group consisting of A, R, N, D, E, Q, G, H, I, L, K, M, F, P, S, T, W, Y and V.

21. The polypeptide of claim 20 wherein said polypeptide is selected from the group consisting of SEQ ID NOs 39 to 57.

22. The polypeptide of claim 1, comprising an amino acid sequence V:
MVFTLEDFVGDWRQTAGYNLDQVLEQGGVSSLFQNLGVSVTPIQRIVLSGENGL RIDIHVIIPYEGLSGDQMGQIERIFRVVYPVDDHHFKVILHYGTLVIDGVTPNMID YFGRPYEGIAVFDGRRITVTGTLWNGNRIIDERLINPDGSLLFRVTINGVTGWRL AERILAX₉ERILA, wherein X₉ is selected from the group consisting of A, R, N, D, E, Q, G, H, I, L, K, M, F, P, S, T, W, Y and V.

23. The polypeptide of claim 22 wherein said polypeptide is selected from the group consisting of SEQ ID NOs 58 to 76.

24. The polypeptide of claim 1, comprising an amino acid sequence VI:
MVFTLEDFVGDWRQTAGYNLDQVLEQGGVSSLFQNLGVSVTPIQKIVLSGENGL KIDIHVIIPYEGLSGDQMGQIEKIFKVVYPVDDHHFKVILHYGTLVIDGVTPNMID YFGRPYEGIAVFDGKKITVTGTLWNGNKIIDERLINPDGSLLFRVTINGVTGWRL X₈ERILA, wherein X₈ is selected from the group consisting of A, R, N, D, E, Q, G, H, I, L, K, M, F, P, S, T, W, Y and V.

25. The polypeptide of claim 24 wherein said polypeptide is selected from the group consisting of SEQ ID NOs 77 to 95.

26. The polypeptide of claim 1, comprising an amino acid sequence VII:
MVFTLEDFVGDWRQTAGYNLDQVLEQGGVSSLFQNLGVSVTPIQKIVLSGENGL RIDIHVIIPYEGLSGDQMGQIERIFRVVYPVDDHHFKVILHYGTLVIDGVTPNMID YFGRPYEGIAVFDGRRITVTGTLWNGNRIIDERLINPDGSLLFRVTINGVTGWRL X₈ERILA, wherein X₈ is selected from the group consisting of A, R, N, D, E, Q, G, H, I, L, K, M, F, P, S, T, W, Y and V.

27. The polypeptide of claim 26 wherein said polypeptide is selected from the group consisting of SEQ ID NOs 96 to 114.

28. A polypeptide comprising a sequence selected from the group consisting of SEQ ID NOs 1 to 114.

29. A polynucleotide encoding the polypeptide selected from any one of the claims 1 to 28.

30. A kit for luminescence assays comprising the polypeptide of any one of claims 1-28.

31. The kit of claim 30, further comprise a substrate for the polypeptide.

32. The kit of claim 31, wherein the substrate is selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.

33. A method of producing luminescence comprising contacting the polypeptide of any one of claims 1-28 with a substrate for the polypeptide.

34. The method of claim 33, wherein the substrate is selected from the group consisting of furimazine, coelenterazine, bisdeoxycoelenterazine, coelenterazine 400a, coelenterazine cp, coelenterazine f, coelenterazine fcp, coelenterazine h, coelenterazine hcp, coelenterazine i, coelenterazine ip, methyl coelenterazine, coelenterazine n, hydrofurimazine and fluorofurimazine.


SEQUENCE LISTING (120 sequences)

SEQ ID NO: 1 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLAERILA

SEQ ID NO: 2 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLRERILA

SEQ ID NO: 3 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLNERILA

SEQ ID NO: 4 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLDERILA

SEQ ID NO: 5 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLEERILA

SEQ ID NO: 6 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLQERILA

SEQ ID NO: 7 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLGERILA

SEQ ID NO: 8 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLHERILA

SEQ ID NO: 9 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLIERILA

SEQ ID NO: 10 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLLERILA

SEQ ID NO: 11 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLKERILA

SEQ ID NO: 12 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLMERILA

SEQ ID NO: 13 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLFERILA

SEQ ID NO: 14 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLPERILA

SEQ ID NO: 15 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLSERILA

SEQ ID NO: 16 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLTERILA

SEQ ID NO: 17 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLWERILA

SEQ ID NO: 18 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLYERILA

SEQ ID NO: 19 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLVERILA

SEQ ID NO: 20 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLAERILA

SEQ ID NO: 21 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLRERILA

SEQ ID NO: 22 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLNERILA

SEQ ID NO: 23 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLDERILA

SEQ ID NO: 24 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLEERILA

SEQ ID NO: 25 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLQERILA

SEQ ID NO: 26 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLGERILA

SEQ ID NO: 27 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLHERILA

SEQ ID NO: 28 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLIERILA

SEQ ID NO: 29 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLLERILA

SEQ ID NO: 30 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLKERILA

SEQ ID NO: 31 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLMERILA

SEQ ID NO: 32 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLFERILA

SEQ ID NO: 33 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLPERILA

SEQ ID NO: 34 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLSERILA

SEQ ID NO: 35 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLTERILA

SEQ ID NO: 36 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLWERILA

SEQ ID NO: 37 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLYERILA

SEQ ID NO: 38 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFRVILHYGTLVIDGVTPNMIDYFGRPYEGIAVFDGKKITVTGTLWNGNRIIDERLINPDGSLLFRVTINGVTGWRLVERILA

SEQ ID NO: 39 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLAERILA

SEQ ID NO: 40 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLRERILA

SEQ ID NO: 41 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLNERILA

SEQ ID NO: 42 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLDERILA

SEQ ID NO: 43 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLEERILA

SEQ ID NO: 44 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLQERILA

SEQ ID NO: 45 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLGERILA

SEQ ID NO: 46 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLHERILA

SEQ ID NO: 47 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLIERILA

SEQ ID NO: 48 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLLERILA

SEQ ID NO: 49 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLKERILA

SEQ ID NO: 50 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLMERILA

SEQ ID NO: 51 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLFERILA

SEQ ID NO: 52 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLPERILA

SEQ ID NO: 53 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLSERILA

SEQ ID NO: 54 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLTERILA

SEQ ID NO: 55 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLWERILA

SEQ ID NO: 56 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLYERILA

SEQ ID NO: 57 - protein, 171 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGKKITVTGTLWNGNRIIDERLINPDGSLLFRVTINGVTGWRLVERILA

SEQ ID NO: 58 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAAERILA

SEQ ID NO: 59 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILARERILA

SEQ ID NO: 60 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILANERILA

SEQ ID NO: 61 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILADERILA

SEQ ID NO: 62 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAEERILA

SEQ ID NO: 63 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAQERILA

SEQ ID NO: 64 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAGERILA

SEQ ID NO: 65 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAHERILA

SEQ ID NO: 66 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAIERILA

SEQ ID NO: 67 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILALERILA

SEQ ID NO: 68 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAKERILA

SEQ ID NO: 69 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAMERILA

SEQ ID NO: 70 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAFERILA

SEQ ID NO: 71 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAPERILA

SEQ ID NO: 72 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILASERILA

SEQ ID NO: 73 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILATERILA

SEQ ID NO: 74 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAWERILA

SEQ ID NO: 75 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAYERILA

SEQ ID NO: 76 - protein, 177 aa, Artificial Sequence
  Synthetic Peptide
MVFTLEDFVGDWRQTAGYNLDQVLEQGGVSSLFQNLGVSVTPIQRIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILAVERILA

SEQ ID NO: 77 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLAERILA

SEQ ID NO: 78 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLRERILA

SEQ ID NO: 79 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLNERILA

SEQ ID NO: 80 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLDERILA

SEQ ID NO: 81 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLEERILA

SEQ ID NO: 82 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLQERILA

SEQ ID NO: 83 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLGERILA

SEQ ID NO: 84 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLHERILA

SEQ ID NO: 85 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLIERILA

SEQ ID NO: 86 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLLERILA

SEQ ID NO: 87 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLKERILA

SEQ ID NO: 88 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLMERILA

SEQ ID NO: 89 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLFERILA

SEQ ID NO: 90 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLPERILA

SEQ ID NO: 91 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLSERILA

SEQ ID NO: 92 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLTERILA

SEQ ID NO: 93 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLWERILA

SEQ ID NO: 94 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLYERILA

SEQ ID NO: 95 - protein, 171 aa, Artificial Sequence
  Synthetic Peptide
MVFTLEDFVGDWRQTAGYNLDQVLEQGGVSSLFQNLGVSVTPIQKIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLVERILA

SEQ ID NO: 96 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLAERILA

SEQ ID NO: 97 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLRERILA

SEQ ID NO: 98 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLNERILA

SEQ ID NO: 99 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLDERILA

SEQ ID NO: 100 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLEERILA

SEQ ID NO: 101 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLQERILA

SEQ ID NO: 102 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLGERILA

SEQ ID NO: 103 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLHERILA

SEQ ID NO: 104 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLIERILA

SEQ ID NO: 105 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLLERILA

SEQ ID NO: 106 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLKERILA

SEQ ID NO: 107 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLMERILA

SEQ ID NO: 108 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLFERILA

SEQ ID NO: 109 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLPERILA

SEQ ID NO: 110 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLSERILA

SEQ ID NO: 111 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLTERILA

SEQ ID NO: 112 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLWERILA

SEQ ID NO: 113 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLYERILA

SEQ ID NO: 114 - protein, 171 aa, Artificial Sequence
  Synthetic Peptide
MVFTLEDFVGDWRQTAGYNLDQVLEQGGVSSLFQNLGVSVTPIQKIVLSGENGLRIDIHVIIPYEGLSGDQMGQIERIFRVVYPVDDHHFKVILHYGTLVIDGVTPNMIDYFGRPYEGIAVFDGRRITVTGTLWNGNRIIDERLINPDGSLLFRVTINGVTGWRLVERILA

SEQ ID NO: 115 - protein, 171 aa, Artificial Sequence
  Synthetic Peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 116 - protein, 171 aa, Artificial Sequence
  Synthetic Peptide
MVLTLEDFVGDWRQTDGYNQAQVLEQGGLTSLFQNLGVSVTPIQRIVLSGENGLKIDIHVIIPYEGLSGDRMGQIEKIFKVVYPVDDHHFKVILHYGTLVIDGVTPNMIDYFGQPYEGIAVFDGKKITVTGTLWNGNKIIDERRINPDGSLLFRVTINGVTGWRLSERILA

SEQ ID NO: 117 - protein, 171 aa, Artificial Sequence
  Synthetic Peptide
MVFTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIIDERLINPDGSLLFRVTINGVTGWRLHERILA

SEQ ID NO: 118 - DNA, 516 bp, Artificial Sequence
  Synthetic DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 119 - DNA, 516 bp, Artificial Sequence
  Synthetic DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgggcgaacgcattctggcgtaa

SEQ ID NO: 120 - DNA, 516 bp, Artificial Sequence
  Synthetic DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgcgcgaacgcattctggcgtaa